(19) United States
(12) Patent Application Publication
(10) Pub. No.: US 2003/0120116 A1
(43) Pub. Date: Jun. 26, 2003

(54) Fixed-bed Raney-type catalysts

(76) Inventors: Daniel Ostgard, Kleinostheim (DE); Andreas Freund, White Plains, NY (US); Claus Rehren, Aschaffenburg (DE); Monika Berweiler, Maintal (DE); Gunter Stephani, Gosserkmannsdorf (DE)

Correspondence Address: SMITH, GAMBRELL & RUSSELL, LLP, SUITE 3100, PROMENADE II, 1230 PEACHTREE STREET, N.E., ATLANTA, GA, 30309-3592, US

(21) Appl. No.: 10/309,919

(22) Filed: Dec. 4, 2002

Related U.S. Application Data
(63) Continuation of application No. 09/611,954, filed on Jul. 6, 2000, now abandoned.
(60) Provisional application No. 60/142,773, filed on Jul. 8, 1999.

(51) Int. Cl. B01J 25/02; C 07C 2 9/36

(52) U.S. Cl. 564/418; 502/301; 502/332; 502/346; 502/302

(57) ABSTRACT

Fixed-bed Raney-type catalysts are made from metal alloy fibers and/or flakes previously fabricated according to the crucible metal extraction method and then tablated, pressed in mats and/or located into a cartridge. The catalysts are used for the hydrogenation, dehydrogenation, isomerization, reductive alkylation, reductive amination, and/or hydration of organic compounds.

### REFERENCE TO A RELATE APPLICATION

This application claims the priority benefit of provisional application 60/142,773 filed Jul. 8, 1999 which is relied on and incorporated herein by reference.

### INTRODUCTION AND BACKGROUND

The present invention relates to fixed bed Raney-type catalysts.

Activated metal catalysts are known in the filed of chemical engineering as Raney catalysts. They are used, largely in powder form, for a large number of hydrogenation, dehydrogenation, isomerization, reductive alkylation, reductive amination and hydration reactions of organic compounds. These powered catalysts are prepared from an alloy of a catalytically-active metal, also referred to herein as a catalyst metal, with a further alloying component which is soluble in alkalis. Mainly nickel, cobalt, copper, or iron are used as catalyst metals. Aluminum is generally used as the alloying component which is soluble in alkalis, but other components may also be used, in particular zinc and silicon or mixtures of these with aluminum.

These so-called Raney alloys are generally prepared by the ingot casting process. In that process a mixture of the catalyst metal and, for example, aluminum are first melted and casted into ingots. Typical alloy batches on a production scale amount to about ten to one hundred kg per ingot. According to DE 21 59 736 cooling times of up to two hours were obtained. This corresponds to an average rate of cooling of about 0.2/s. In contrast to this, rates of 102 to 106 K/s are achieved in processes where rapid cooling is applied (for example an atomizing process). The rate of cooling is affected in particular by the particle size and the cooling medium (see Materials Science and Technology edited by R. W. Chan, P. Haasen, E. J. Kramer, Vol 15, Processing of Metals and Alloys, 1991, VCH-Verlag Weinheim, pages 57 to 110). A process of this type is sued in EP 0 437 788 B 1 in order to prepare a Raney alloy powder. In that process the molten alloy at a temperature of 50 to 500° C. above its melting point is atomized and cooled using water and/or a gas.

To prepare a catalyst, the Raney alloy is first finely milled if it has not been produced in the desired powder form during preparation. Then the aluminum is entirely or partly removed by extraction with alkalis such as, for example, caustic soda solution. This activates the alloy powder. Following extraction of the aluminum the alloy powder has a high specific surface area (BET), between 20 and 100 m²/g, and is rich in active hydrogen. The activated catalyst powder is pyrophoric and stored under water or organic solvents or is embedded in organic compounds which are solid at room temperature.

Powered catalysts have the disadvantage that they can be used only in a batch process and, after the catalytic reaction, have to be separated from the reaction medium by costly sedimentation and/or filtration. Therefore a variety of processes for preparing molded items which lead to activated metal fixed-bed catalysts after extraction of the aluminum have been disclosed. Thus, for example, coarse particulate Raney alloys, i.e., Raney alloys which have only been coarsely milled, are obtainable and these can be activated by a treatment with caustic soda solution. Extraction and activation then occurs only in a surface layer the thickness of which can be adjusted by the conditions used during extraction.

A substantial disadvantage of catalysts prepared by these prior methods are the poor mechanical stability of the activated outer layer. Since only this outer layer of the catalyst is catalytically active, abrasion leads to rapid deactivation and renewed activation of deeper lying layers of alloy using caustic soda solution then leads at best to partial reactivation.

Patent application EP 0 648 534 B1 describes shaped, activated Raney metal fixed-bed catalysts and their preparation. This is intended to avoid the disadvantages described above, e.g., the poor mechanical stability resulting from activating an outer layer. To prepare these catalysts, a mixture of powders of a catalyst alloy and a binder are used, where in the catalyst alloys each contain at least one catalytically active catalyst metal and an extractable alloying component. The pure catalyst metals or mixtures thereof which do not contain extractable components are used as binder. The use of the binder in an amount of 0.5 to 20 weight percent with respect to the catalyst alloy, is essential in order to achieve sufficient mechanical stability after activation.

After shaping the catalyst alloy and the binder with conventional shaping aids and pore producers, the freshly prepared items which are obtained are calcined at temperatures below 850° C. As a result of sintering processes with the finely divided binder, this produces solid compounds between the individual granules of the catalyst alloy. These compounds, in contrast to catalyst alloys, are non-extractable or only extractable to a small extent so that a mechanically stable structure is obtained even after activation.

However, the added binder has the disadvantage that it is substantially catalytically inactive and thus the number of active centers in the activated layer is reduced. In addition, the absolutely essential use of a binder means that only a restricted range of amounts of pore producers can be used without endangering the strength of the shaped item. For this reason, the bulk density of these catalysts cannot be reduced to a value of less than 1.9 kg per liter without incurring loss of strength. This leads to a considerable economic disadvantage when using these catalysts in industrial processes. In particular when using more expensive catalysts alloys, for example cobalt alloys, the high bulk density leads to a high investment per reactor bed, which is, however partly compensated for by the high activity and long-term stability of these catalysts. In certain cases, the high bulk density of the catalyst also requires a mechanically reinforced reactor structure.

An object of the present invention is therefore to provide an activated base metal catalysts from metallic fibers and flakes which largely avoids the disadvantages of known fixed-bed catalysts.

### SUMMARY OF THE INVENTION

The above and other objects of the invention can be achieved by fixed bed Raney type catalysts which are characterized in that they are made from metal alloy fibers and/or flakes previously fabricated according to the crucible metal extraction method and then tablated, pressed in mats, and/or loaded into a cartridge.

A further feature of the present invention is a method to produce the fixed-bed Raney-type catalysts wherein metal alloy fibers and/or flakes are produced by the crucible melt extraction method and afterwards tablated, pressed into mats, and/or loaded into a cartridge.

In a preferred form of the invention the fixed-bed Raney-type catalysts can be activated.

### DETAILED DESCRIPTION OF THE INVENTION

In describing the more particular aspects of the invention, it should be noted that the invention can be carried out by producing fibers or flakes out of the desired metals and/or alloys by the crucible melt extraction method and performing any necessary activation procedures in order to make the catalyst. The major advantages of this invention are that the fibers and flakes are rapidly cooled so as to produce the desired small phase domains in these Raney alloys, and unlike powders, these materials are easier to separate from the reaction mixture. They could either be loaded into a cartridge arrangement to perform its catalytic reaction, or they could be pressed into mats, tablets, and other commonly used forms that are readily useable as fixed bed catalysts.

In a preferred way, the fibers and flakes made for this patent are prepared according to DE 197 11 764 A1 by the crucible melt extraction method. This reference is relied on and incorporated herein by reference. In this method, a rectangular crucible of melted alloy is situated below a spinning water cooled copper wheel outfitted with specially designed notches on its surface. The crucible is then slowly raised so that the spinning wheel barely makes contact. In this fashion, the wheel skims the molten alloy while the molten material quickly collects and solidifies in the wheels notched surface before being rapidly released into a collection area. The result is a rapidly cooled material with a controllable shape. Depending on the design of the notches on the spinning wheel, the finished material would either be flakes or fibers of a certain length and diameter.

Raney catalyst alloys are typically obtained from a melt of the catalyst metal and aluminum. Nevertheless, different phase structures may be obtained from identical microscopic compositions, depending on how and how fast the molten material is cooled. A coarse phase structure with large phase domains is generally produced when casting ingots, due to the low rate of cooling. A substantially finer structure is produced, however when a more rapid cooling procedure is used. The rate of cooling required can be determined by a person skilled in the art by appropriate trials. As a guideline for the rates of cooling required, cooling times from the melting point to less than 700° C. of less than two minutes may be mentioned. This corresponds to rates of cooling of at 5 K/s.

The bulk density of the resulting fixed bed catalyst is also important for highly active catalysts. This should be less than 2.0 kg/l. If the bulk density is more than 2.0 kg/l, the catalysts are too compact and thus less active. The ratio by weight of catalyst metal to extractable alloying component in the catalyst alloys is, as is conventional with Raney alloys, in the range from 20:80 to 80:20. Catalysts according to the invention may also be doped with other metals in order to have an effect on the catalytic properties. The purpose of this type of doping, is for example, to improve the selectivity in a specific reaction. Doping metals are frequently also called promoters. The doping or promoting of Raney catalyst is described for example in U.S. Pat. No. 4,153,578 and DE-AS 21 01 856 in DE-OS 21 00 373 and in the DE-AS 2053799. All these documents are incorporated herein by reference.

In principle, any known metal alloys with extractable elements such as aluminum zinc and silicon may be used for the present invention. Suitable promoters are transition elements in groups of 3B to 7B and 8 and group 1B of the Periodic Table of Elements and also the rare-earth metals. They are also used in an amount of up to 20 wt %, with respect of the total weight of catalyst. Chromium, manganese, iron, cobalt, vanadium, tantalum, titanium, tungsten, and/or molybdenum and metals from platinum group are preferably used as promoters. They are expediently added as alloying constituents in the catalyst alloy. In addition, promoters with a different extractable metal alloy, in the form of a separable metal powder, may be used or the promoters may be applied later to the catalyst material. Later application of promoters may be performed either after calcination or after activation. Optimum adjustment of the catalyst properties to the particular catalyst process is thus possible.

The Raney type catalysts made here were either loaded into a cartridge and activated with a caustic soda solution, or they were pressed into forms such as tablets and mats. The formed material was made optionally with a binder, such a Ni powder, calcined in air, and activated with a caustic soda solution. For this purpose, for example, a 20% strength caustic soda solution heated to 80° C. may be used. The Raney type catalyst precursors resulting from calcination are also very important with regard to economic viability of invention. They are not pyrophoric and can be handled and transported without difficulty. Activation can be performed by the user shortly before use. Storage under water or organic solvents or embedding in organic compounds is not required for the catalyst precursors.

The fixed-bed Raney-type catalysts according the invention can be used for the hydrogenation, dehydration, isomerization, reductive alkylation, reductive animation, and/or hydration reaction of organic compounds.

Comparison Exhibit 1

A free-flowing, pelletilizible catalyst mixture was prepared in accordance with the instructions in EP 0 648 534 A1 for a comparison catalyst consisting of 1000 g of 53% Ni and 47% A1 alloy powder, 150 g of pure nickel powder (?99% Ni, and d50=21 μm), and 25 g of ethylene bis-stearoylamide whilst adding about 150 g of water. Tablets with the diameter of 4 mm and a thickness of 4 mm were compressed from this mixture. The shaped items were calcined for 2 h at 700° C. The tablets were activated in 20% strength caustic soda solution for 2 hours at 80° C. after calcination

Comparison Exhibit 2

A free-flowing, pelletilizible catalyst mixture was prepared in accordance with the instructions in EP 0 648 534 A1 for a comparison catalyst consisting of 1000 g of 53% Ni and 47% A1 alloy powder, 150 g of pure nickel powder (?99% Ni, and d50=21 μm), and 25 g of ethylene bis-stearoylamide whilst adding about 150 g of water. Tablets with the diameter of 10 mm and a thickness of 6 mm were compressed from this mixture. The shaped items were calcined for 2 h at 700° C. The tablets were activated in 20% strength caustic soda solution for 2 hours at 80° C. after calcination.

### EXAMPLE 1

A mixture was made with 20 grams of 50% NI:50% A1 fibers (5 mm long with the diameter of 100 μm) and 1 gram of 87%:13% A1 (5 mm long with a diameter of 30-50 μm). Tablets with the diameter of 10 mm and a thickness of 6 mm were compressed from this mixture. The shaped items were calcined for 2 h at 700° C. The tablets were activated in 20% strength caustic soda solution for 1.5 hours at 80° C. after calcination.

### EXAMPLE 2

A mixture was made with 20 grams of 50%NI:50% A1 fibers (5 mm long with the diameter of 100 μm) and 2.8 grams of Ni powder (d50=21 μm). Tablets with the diameter of 10 mm and a thickness of 6 mm were compressed from this mixture. The shaped items were calcined for 2 h at 700° C. The tablets were activated in 20% strength caustic soda solution for 1.5 hours at 80° C. after calcination.

### EXAMPLE 3

A mixture was made with 35 grams of 50% Ni:50% A1 fibers (5 mm long with the diameter of 100 μM) and 0.89 gram of Ni powder (d50=21 μm). Tablets with the diameter of 4 mm and a thickness of 4 mm were compressed from this mixture. The shaped items were calcined for 2 h at 700° C. The tablets were activated in 20% strength caustic soda solution for 1.5 hours at 80° C. after calcination.

### EXAMPLE 4

A mixture was made with 35 grams of 50% Ni:50% A1 fibers (5 mm long with the diameter of 100 μm) and 0.89 gram of Zn powder (d50?60 μm). Tablets with the diameter of 4 mm and a thickness of 4 mm were compressed from this mixture. The shaped items were calcined for 2 h at 700° C. The tablets were activated in 20% strength caustic soda solution for 1.5 hours at 80° C. after calcination.

### EXAMPLE 5

A teflon basket was filled with 7 grams of 50% Ni:50% A1 fibers (5 mm long with the diameter of 100 μm) and activated in 20% strength caustic soda solution for 1.5 hours at 80° C.

### APPLICATION EXAMPLE 1

The catalytic activity of the catalyst from comparison examples 1 and 2 and from examples 1 to 5 were compared during the hydrogenation of nitrobenzene. For this purpose, 100 g of nitrobenzene and 100 g of ethanol were placed in a stirred autoclave with a capacity of 0.5 l, fitted with a gas stirrer. 10 g of the catalyst being investigated were suspended each time in the stirred autoclave using a catalyst basket so that the catalyst material was thoroughly washed by the reactant/solvent mixture, and hydrogen was introduced. Hydrogenation was performed at a hydrogen pressure of 40 bar and a temperature of 150° C. The initiation temperature and the rate of hydrogen consumption were determined. The results are given in table 1. As a check, samples were withdrawn after 1, 2, 3, 4, and 5 h and analyzed using gas chromatography.

Further variations and modifications will be apparent to those skilled in the art from the foregoing and are intended to be encompassed by the claims appended hereto.

German application 199 31 316.4 is relied on and incorporated herein by reference.

TABLE 1
The hydrogenation of Nitrobenzene to Aniline.

| | Formed | | Bulk | Initiation | Rate of Hydrogen | Rate of H₂cons. |
|---|---|---|---|---|---|---|
| | Body | | Density, | Temperature, | Consumption, | ml H₂/(h) |
| Catalyst | Size mm | Binder | kg/l | ° C. | ml H2/(h) (g of cat.) | (ml cat.) |
| CE1 | 4 × 4 | Ni powder | 2.3 | 125 | 1.3 | 2.99 |
| CE2 | 10 × 6 | Ni powder | 1.6 | 141 | 0.4 | 0.64 |
| E1 | 10 × 6 | 87% Ni | 1.6 | 112 | 2.26 | 3.62 |
| | | 13% Al | | | | |
| | | fibers | | | | |
| E2 | 10 × 6 | Ni powder | 1.6 | 135 | 0.83 | 1.33 |
| E3 | 4 × 4 | Ni powder | 1.4 | 120 | 2.74 | 3.84 |
| E4 | 4 × 4 | Zn powder | 1.3 | 117 | 3.68 | 4.78 |
| E5 | — | None | — | 140 | 1.9 | — |

We claim:

1. A fixed-bed Raney-type catalyst comprising:
at least one metal alloy fiber or flake formed by crucible metal extraction.

2. The catalyst according to claim 1 which is in tablet or mat form.

3. The catalyst according to claim 1 which is loaded into a cartridge.

4. The catalyst according to claim 1 wherein the catalyst has desired small phase domains as a result of rapid cooling in the crucible metal extraction.

5. The catalyst according to claim 1 which is obtained from a melt of a catalyst metal and aluminum.

6. The catalyst according to claim 1 wherein the catalyst metal is a member selected from the group consisting of nickel, cobalt, copper and iron.

7. The catalyst according to claim 5 wherein the catalyst metal is nickel.

8. The catalyst according to claim 1 which has a bulk density of less than 2 kg/l.

9. The catalyst according to claim 1 wherein the metal alloy consists of a catalytic metal and an extractable alloying component.

10. The catalyst according to claim 9 wherein the ratio of catalytic metal to extractable alloying component is from 20:80 to 80:20.

11. The catalyst according to claim 9 wherein the extractable alloying component is a member selected from the group consisting of aluminum, zinc and silicon.

12. The catalyst according to claim 1 further comprising a promoter.

13. The catalyst according to claim 12 wherein the promoter is a member selected from the group consisting of the transition elements in Group 3B to 7B and 8 and Group 1B of the Periodic Table of Elements.

14. The catalyst according to claim 12 wherein the promoter is a rare earth element.

15. The catalyst according to claim 1 where the fiber has an average length of 5mm.

16. The catalyst according to claim 1 wherein the fiber has an average diameter of 100 microns.

17. A method of producing fixed-bed Raney-type catalysts comprising forming metal alloy fibers or flakes from a melt of a catalytic metal and an extractable alloying component as a molten material in a crucible, rapidly cooling the molten material under controlled conditions so as to form the desired fiber or flake.

18. The method according to claim 17 further comprising cooling molten material at a rate from the melting point to less than 700° in less than two minutes.

19. The method according to claim 17 further comprising introducing a promoter into the molten material.

20. The method according to claim 17 further comprising activating said fibers or flakes.

21. A method for the hydrogenation of nitrobenzene comprising contacting said nitrobenzene with hydrogen in the presence of the catalyst of claim 1.
